(19) United States
(12) Patent Application Publication
(10) Pub. No.: US 2008/0199538 A1
(43) Pub. Date: Aug. 21, 2008

(54) Compound to enhance lucid erotic dreams

(76) Inventor: Yronne Filopelrick, Fayetteville, NC (US)

Correspondence Address: GREENBERG & LIEBERMAN, LLC, 2141 WISCONSIN AVE, N.W., SUITE C-2, WASHINGTON, DC, 20007, US

(21) Appl. No.: 11/306,741

(22) Filed: Jan. 10, 2006

(51) Int. Cl. A61K 36/67; A61K 36/42; A61K 36/53; A61K 36/28; A61K 33/06; A61K 31/197; A61K 31/455; A61K 31/404; A61K 31/14; A61K 31/4415; A61K 31/195; A61K 31/4985; A61K 31/714; A61K 31/375; A61P 25/20

(52) U.S. Cl. 424/682; 424/734; 424/758; 424/745; 424/764; 514/563; 514/355; 514/416; 514/667; 514/345; 514/567; 514/249; 514/52; 514/562; 514/561; 514/474

(57) ABSTRACT

A compound that contains herbal supplements to increase the frequency and lucidity of a person's erotic or sexual dreams. The compound can be used by anyone wishing to enhance their lucid erotic dreams, or may also be taken by people suffering from jet lag or insomnia. Modes of delivery are by capsule, tablet, soft gel, transdermal patch, liquid, and gum.

### FIELD OF THE INVENTION

The present invention is a pill designed to enhance the lucid erotic dreams of a user.

### BACKGROUND OF THE INVENTION

It is already known that there exists medication to help a person suffering from insomnia or other sleep disorders; there are also medications and therapies on the market to help people suffering from sexual disorders. It is also known that there are methods of improving the lucidity of dreams. However, heretofore, there has been no such invention that would be able to combine the aforementioned aspects, that would a) aid in the recall, frequency and lucidity of dreams; b) promote sleep; c) and encourage a user to have more erotic dreams as a way of releasing sexual energy and sexual tension and fulfilling fantasies that might not otherwise be possible in daily life.

Therefore, a need has been established for a compound that can enhance the lucidity of dreams, enhance the frequency of erotic dreams, and serve as a sleeping aid to promote sleep in a user. Relevant art to the present invention includes several publications and patents. The patents discussed do not incorporate all of the elements that make the present invention unique.

The following patents or publications are compounds that aim to alleviate sleep (and/or) sexual disorders or improve sleep and mental performance.

European Publication 1241170 A2, filed Feb. 27, 2002, discloses therapeutic compounds for the treatment of sexual disorders.

U.S. Pat. No, 4,833,154 issued to Jean-Louis et al. on May 23, 1989, is a sleeping pill to alleviate insomnia.

U.S. Pat. No. 6,586,478 B2 issued to Ackman et al. are methods and compositions for improving sleep.

US Publication 2004/0254202 A1 filed by Ebel on Dec. 16, 2004 is a method of promoting sleep using topical administration of vasoactive agents.

WO Publication 03/003981 published on Jan. 16, 2003 are compositions for improving mental performance.

US Publication 2005/0177192 filed by Rezai et al. on Aug. 11, 2005, discloses a method of neurostimulation for affecting sleep disorders.

The following aim to aid in the detection, recording and/or recall of dreams.

U.S. Pat. No. 4,735,199 issued to DiLullo on Apr. 5, 1998, is a dream detection method and system through the monitoring of a physical subject.

U.S. Pat. No. 4,863,259 issued to Schneider et al. on Sep. 5, 1989, is a sleep state detector.

U.S. Pat. No. 5,518,79 issued to Raynie et al. on Sep. 3, 1996, is a dream state teaching machine.

U.S. Pat. No. 6,540,664 issued to Blair on Apr. 1, 2003, is an apparatus to facilitate the analysis of dream activity.

US Publication 2004/0192296 A1 filed by Lemmon on Sep. 30, 2004 is a device for recording dream recollections.

The following aim to help induce lucidity during dreaming.

U.S. Pat. No. 5,507,716 issued to LaBerge et al. on Apr. 16, 1996, discloses equipment and methods used to induce lucid dreaming.

US Publication 2004/0266659 A1 filed by LaBerge et al. on Dec. 30, 2004 discloses substances to enhance recall and lucidity during dreaming. However, it makes no reference to enhancing (specifically) the erotic lucid dreams of a user.

Unlike the present invention, which is a combination of various objectives, the aforementioned patents are limited in scope in comparison to the goals of the present invention.

### SUMMARY OF THE INVENTION

Lucid dreaming is when a person is fully aware that they are dreaming. They are able to control their dreams and alter the dream course or control all aspects of the dream. They are also able to recall their dream upon waking up. Lucid dreaming generally takes place during REM (Rapid Eye Movement) sleep. Five stages of sleep generally occur during the sleep cycle. As the person descends from stages 1-4, 4 being the deepest sleep, the brain waves/frequency begin to slow down. After about 90 minutes of sleep, REM sleep begins. Only 5-10 minutes are spent in REM sleep before the person goes back into Stage 4 sleep. After another 90 minutes, another REM period starts. The REM lasts longer during this second period. The cycle continues this way, with REM occurring on average every 90 minutes. Vivid dreaming occurs during REM, so the more the person sleeps, the more time is spent in REM and the more likely they are to have a lucid dream.

The present invention is a pill designed to enhance the lucid erotic dreams of a user. It is intended for people 21 and over who cannot experience sexual activity in everyday life for various reasons, such as impotence and injury, or who simply wish to enhance the lucidity, recall and frequency of their erotic dreams. Through the sustained use of the present invention, the lucid sex dreams will become more frequent (1-3 times a week) after 5 to 8 weeks of use.

It is an object of the present invention to increase memory recall after sleep and dreaming; to increase the frequency of lucid erotic dreams in men and women; to also serve as a sleeping pill for those suffering from insomnia and jet lag; to restore brain chemistry to pre-teen states. It is an object of the present invention to lower the stress and tension levels, which affect the ability of the person to remember their dreams. It is an object of the present invention to allow a person to increase their sexual fantasies within their lucid dreams, thereby resulting in an increase of the release of endorphins, which are natural painkillers. It is a further object of the present invention to allow a user to become relaxed by promoting sleep and preventing nightmares.

### DETAILED DESCRIPTION OF THE PREFERRED EMBODIMENT

The present invention is comprised of the following ingredients.

| | Optimal Dosage | Minimal/Maximal Dosage |
|---|---|---|
| | (in Mg, unless | (in Mg, unless |
| Ingredient | otherwise stated) | otherwise stated) |
| B-3 Niacinamide | 250 | 50/1000 |
| B-5 Pantothenic Acid | 50 | 25/500 |
| Choline | 30 | 10/1200 |
| B-6 Pyridoxine | 10 | 1/275 |
| B-9 Folic acid | 400 Mcg | 50 Mcg/800 Mcg |
| B-12 Methylcobatamin | 50 Mcg | 10 Mcg/5000 Mcg |
| L-Methionine | 165 | 25/3100 |
| L-Serine | 410 | 100/500 |
| Calcium | 200 | 10/1500 |
| Magnesium | 100 | 10/1000 |
| DMAE | 100 | 25/351 |
| (Dimethylaminoethanol) | | |
| Vitamin C | 300 | 50/1000 |
| Kava Kava root | 200 | 50/750 |
| L-Phenylalanine | 245 | 50/1000 |
| L-Tyrosine | 240 | 50/500 |
| Melatonin | 1 | 1/3 |
| Pumpkin Seed Oil | 400 | 100/2000 |
| L-Cysteine | 134 | 25/600 |
| Lavender | 400 | 100/1000 |
| Chamomile | 350 | 100/900 |

B-vitamins in general help a person have more vivid dreams:

B-3 vitamin Niacinamide increases the production of sexual hormones, which can trigger sex dreams; it also increases serotonin production.

B-5 vitamin and Choline combine to form acetylcholine, a neurotransmitter, which is excreted from the pons in the brainstem and produces REM.

B-6 vitamin is a co-enzyme involved in the production of proteins and neurotransmitters and the breakdown of amino acids. It is useful for processing amino acid transmitters and hormones such as a) serotonin, b) dopamine, c) melatonin and d) norepinephrine. B-6 also promotes the conversion of the amino acid tryptophan into serotonin and niacinamide (B-3 vitamin).

a) Serotonin is a neurotransmitter playing an important role in the biochemistry of sexuality, appetite, and mood. Low serotonin levels are a factor in depression.

b) Dopamine is commonly associated with the pleasure centers of the brain, and is released upon enjoyable experiences such as eating food and during sexual activity.

c) Melatonin is helpful for falling asleep, and increases non-REM sleep.

d) Norepinephrine governs the fight-or-flight response.

B-9, B-12, and amino acids L-Methionine and L-Serine help synthesize the B-vitamin Choline, which in turn produces acetylcholine.

Calcium and Magnesium help promote serotonin production.

DMAE (Dimethylaminoethanol) is also a B-vitamin that is converted into Choline.

Vitamin C helps metabolize certain amino acids and hormones.

Kava kava root is known to tranquilize, de-stress and take away tension, as well as sharpen one's mind. It is also thought to induce lucid dreaming.

L-Phenylalanine is an amino acid that is converted into tyrosine and subsequently into dopamine.

L-Tyrosine is metabolized into dopamine.

Melatonin increases non-REM sleep and makes it easier to fall asleep. It also has an interesting rebound effect in that it may produce more frequent and vivid dreams.

Pumpkin Seed Oil helps trigger sexual dreams.

Cysteine is an amino acid and helps produce dopamine.

Lavender and Chamomile both have calming and relaxing properties. Lavender may induce erotic dreams and Chamomile soothes and may prevent nightmares. The pill is to be taken twice daily, and there are 60 pills per bottle.

Although various embodiments of the present invention are herein described, it is contemplated that the present invention could be made of Kava kava root, L-Phenylalanine, Niacinamide, pumpkin seed oil and lavender.

Having illustrated the present invention, it should be understood that various adjustments and versions might be implemented without venturing away from the essence of the present invention. The present invention is not limited to the embodiments described above, and should be interpreted as any and all embodiments within the scope of the following claims.

What is claimed is:

1. A compound to enhance lucid erotic dreams for a user, comprising:
50-750 Mg of Kava Kava root;
50-1000 Mg of L-Phenylalanine;
50-1000 Mg of Niacinamide;
100-2000 Mg of pumpkin seed oil; and
100-1000 Mg of Lavender.

2. The compound of claim 1 further comprising:
1-3 Mg of Melatonin;
25-351 Mg of Dimethylaminoethanol;
1-275 Mg of B-6 Pyridoxine;
50-500 Mg of L-Tyrosine;
10-1200 Mg of Choline;
25-500 Mg of B-5 Pantothenic acid;
50-800 Mcg of B-9 Folic Acid; and
10-5000 Mcg of B-12 Methylcobatamin.

3. The compound of claim 1 further comprising:
25-3100 Mg of L-Methionine;
25-600 Mg of L-Cysteine.
100-500 Mg of L-Serine;
100-900 Mg of Chamomile;
50-1000 Mg of Vitamin C;
10-1500 Mg of Calcium; and
10-1000 Mg of Magnesium.

4. A compound to enhance lucid erotic dreams for a user, comprising:
Kava Kava root;
L-Phenylalanine;
Niacinamide;
pumpkin seed oil; and
Lavender.
